(19) United States
(12) Patent Application Publication
(10) Pub. No.: US 2024/0415508 A1
(43) Pub. Date: Dec. 19, 2024

(54) SUTURE CUTTING TOOL

(71) Applicant: Suture Slicer, Inc., Meadowbrook, PA (US)

(72) Inventors: Todd D. Schwartz, Meadowbrook, PA (US); Jake Ganem, Cape Neddick, ME (US)

(21) Appl. No.: 18/743,080

(22) Filed: Jun. 13, 2024

Related U.S. Application Data
(60) Provisional application No. 63/472,595, filed on Jun. 13, 2023.

(51) Int. Cl. A61B 17/04

(52) U.S. Cl. CPC A61B 17/0467

(57) ABSTRACT

A suture cutting tool having a housing, a plunger assembly and a blade. The housing has an inner bore and a main body having an anvil assembly that is positioned at the distal end of the housing, defining a suture slot. The anvil assembly having top surface and a blade slot. The plunger assembly has a plunger rod slidably movable relative to the housing. The blade is coupled to the plunger rod and includes a cutting edge. The blade is movable along with the plunger assembly between at least a first position having the cutting edge within the suture slot to a second position having the cutting edge within the blade slot of the anvil. Thru the movement between the first and second positions, the cutting edge is structurally configured to cut a suture traversing the blade slot.

### CROSS-REFERENCE TO RELATED APPLICATION

This application claims priority from U.S. Prov. Patent App. No. 63/472,595 entitled Suture Cutting Tool, filed Jun. 13, 2023, the entire disclosure of which is hereby incorporated by reference in its entirety.

### BACKGROUND OF THE DISCLOSURE

### 1. Field of the Disclosure

The disclosure relates in general to a medical device, and more particularly, to a suture cutting tool that is configured to cut a suture utilized during surgery or another procedure, typically, with internal suturing, while not limited thereto.

### 2. Background Art

Suturing is a medical technique which involves the stitching together of tissue, typically in a surgery or another medical procedure. One type of suturing is internal suturing which is a medical technique that involves stitching together internal tissues or organs that have been cut or injured during surgery or another medical procedure. Such a procedure is commonly used in various types of surgeries, including those involving the digestive, urinary, and reproductive systems, while not being limited thereto.

Although internal suturing can be effective in promoting healing and reducing the risk of complications, the procedure is not without its challenges. Typically, when completing such a procedure, suture is cut using scissors, or the like. One common problem associated with internal suturing procedures is the risk of tissue damage from the scissors contacting or damaging tissue while handling the scissors, or inadvertently cutting tissue when cutting the suture. In such instances, the damage can lead to infection, bleeding, or even organ failure. In addition, improper handling of scissors or other devices can result in tissue tearing, hernias, and other complications, some of which may require further medical intervention.

### SUMMARY OF THE DISCLOSURE

In an aspect of the disclosure, the disclosure is directed to a suture cutting tool comprising a housing, a plunger assembly and a blade. The housing has a distal end, a proximal end, and an inner bore defined therethrough. The housing further comprises a main body having an anvil assembly, with the anvil assembly positioned at the distal end of the housing, the main body and anvil assembly coupled by a connecting bridge to define a suture slot. The anvil assembly having top surface facing the inner bore and defining a blade slot between a first and second side of the top surface. The plunger assembly has a plunger rod slidably movable within the inner bore of the housing with a first end extending beyond the housing and a second end extendable beyond the inner bore of the housing, The blade is coupled a distal end of the plunger rod and including a cutting edge, the blade being movable along with the plunger assembly between at least a first position having the cutting edge within the suture slot to a second position having the cutting edge within the blade slot of the anvil. Upon movement between the first and second positions, the cutting edge is structurally configured to cut a suture traversing the blade slot within the suture slot.

In some configurations, the cutting edge is precluded from reaching a lower end of a bottom wall of the blade slot.

In some configurations, the blade slot further includes a first wall and a second wall opposite the first wall, the first and second walls being spaced apart from each other and depending from the top surface, wherein the blade is precluded from abutting either of the first and second walls.

In come configurations, the blade defines a thickness and, upon insertion into the blade slot, a distance between the blade and the first wall and the blade and the second wall is less than or equal to the thickness of the blade.

In some configurations, the blade has a thickness of 0.004″ and the blade slot has a width of 0.010″, with the blade substantially bisecting therebetween.

In some configurations, the tool further includes a return spring having a first end interfacing with the housing and a second end interfacing with the plunger rod. The return spring forces the blade away from the second position.

In some configurations, in the first position, the cutting edge is positioned within the inner bore of the housing.

In some configurations, the tool further includes at least one longitudinal slot defined into the inner bore of the housing terminating at the proximal end of the housing. Additionally, at least one outer surface tab on an outer surface of the plunger rod, the at least one outer surface tab slidably movable within the at least one longitudinal slot of the housing. The at least one longitudinal slot capturing the at least one outer surface tab so as to constrain relative rotation of the plunger rod to the housing.

In some configurations, the at least one outer surface tab and the at least one longitudinal slot are configured to increasingly constrain relative rotation of the plunger rod to the housing as the blade is directed from the first position to the second position.

In some configurations, the at least one outer surface tab comprises four outer surface tabs and the at least one longitudinal slot comprises four longitudinal slots, space apart on the inner bore of the housing.

In some configurations, the at least one outer surface tab has a lower end tab wall that interfaces with distal stop that precludes further movement in the direction of the second end.

In some configurations, the outer surface of the plunger rod further includes a plurality of protrusions extending outwardly therefrom, the plurality of protrusions interfacing with the inner bore so as to improve alignment of the blade relative to the blade slot as the blade is directed to the second position.

In some configurations, at least one of the plurality of protrusions contacts the inner bore as the blade is directed toward the second position prior to the cutting blade entering into the blade slot, while being spaced apart from the inner bore toward the first position.

In some configurations, the plunger rod defines a longitudinal axis of travel within the inner bore, with the top surface of the anvil assembly being substantially perpendicular thereto.

In some configurations, the blade slot includes a first sidewall and a second sidewall, with the first sidewall and second sidewall being parallel to each other and perpendicular to the top wall.

In some configurations, the housing comprises two components coupled end to end, the first component being the main body and the second component being a sleeve, with the plunger rod extending through the main body and the sleeve.

In some configurations, the connecting bridge further includes a facing wall, with the blade slot meeting the facing wall.

In some configurations, the blade includes a cutting edge, with the cutting edge being substantially perpendicular to a longitudinal axis of the plunger rod.

In another aspect of the disclosure, the disclosure is directed to a method of using a suture cutting tool comprising the steps of: providing the suture cutting tool as disclosed herein; placing a suture into the suture slot and across the blade slot; directing the plunger rod from the first position to the second position; and cutting the suture as the blade passes beyond the top wall.

In some configurations, the suture is maintained outside of the blade slot during the step of cutting.

In another aspect of the disclosure, the disclosure is directed to a suture cutting tool comprising a housing, an anvil and a plunger rod. The housing has a proximal end and a distal end, with an internal bore extending therethrough. The distal end defining a suture slot that is transverse to the internal bore, with the housing terminating with an anvil assembly opposite the suture slot. The anvil having a top wall extending thereacross with a blade slot depending from the top wall. The plunger rod is slidably positionable within the internal bore with a first end extending outside of the proximal end and a second end having a blade that is insertable into the suture slot and a cutting edge of which is insertable into the blade slot.

In some configurations, the blade slot includes a first sidewall and a second sidewall, the first sidewall and second sidewall being spaced apart from the cutting edge of the blade throughout the relative positions of the blade within the blade slot.

In yet another aspect of the disclosure, the disclosure is directed to a method of using a suture cutting tool comprising the steps of: providing the suture cutting tool, the suture cutting tool having a housing with an anvil assembly proximate the distal end thereof, the anvil assembly having a top surface and a blade slot formed therein, and a suture slot defined between the proximal end of the housing and the top surface of the anvil assembly, and, a plunger rod slidably movable relative to the housing, a blade attached to the plunger rod proximate the second end thereof, the blade having a cutting edge slidably directable through a portion of the suture slot and the blade slot; placing a suture into the suture slot and across the blade slot; directing the plunger rod from the first position to the second position; and cutting the suture as the blade passes beyond the top wall.

In some configurations, the step of cutting occurs at the top wall, with the suture substantially remaining outside of the blade slot.

In some configurations, the method includes the step of: removing the cutting edge from within the blade slot after the step of cutting.

In some such configurations, the method includes the step of: removing the cutting edge from within the suture slot after the step of removing the cutting edge from within the blade slot.

### BRIEF DESCRIPTION OF THE DRAWINGS

The disclosure will now be described with reference to the drawings wherein:

FIG. 1 of the drawings is a perspective view of a configuration of the suture cutting tool of the present disclosure, showing, in particular, the blade in a retracted configuration;

FIG. 2 of the drawings is a cross-sectional perspective view of the configuration of the suture cutting tool as shown in FIG. 1;

FIG. 3 of the drawings is a perspective view of a configuration of the suture cutting tool of the present disclosure, showing, in particular, the blade in an extended configuration;

FIG. 4 of the drawings is a cross-sectional perspective view of the configuration of the suture cutting tool as shown in FIG. 3;

FIG. 5 of the drawings is an exploded view of a configuration of the suture cutting tool;

FIG. 6 of the drawings is a perspective view of the housing of the suture cutting tool of the present disclosure;

FIG. 7 of the drawings is a cross-sectional view of the housing of the suture cutting tool of the present disclosure;

FIG. 8 of the drawings is a partial cross-sectional perspective view of the housing of the suture cutting tool of the present disclosure;

FIG. 9 of the drawings is a partial perspective view of the housing of the suture cutting tool of the present disclosure, showing in particular, the anvil assembly thereof;

FIG. 10 of the drawings is a cross-sectional perspective view of the sleeve of the suture cutting tool of the present disclosure;

FIG. 11 of the drawings is a partially exploded view of the plunger assembly of the suture cutting tool of the present disclosure;

FIG. 12 of the drawings is a partial perspective view of the plunger rod of the plunger assembly of the present disclosure;

FIG. 13 of the drawings is a partial perspective view of the plunger rod and return spring of the plunger assembly of the present disclosure; and

FIG. 14 of the drawing is a partial perspective view of the plunger rod and blade of the plunger assembly of the present disclosure.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

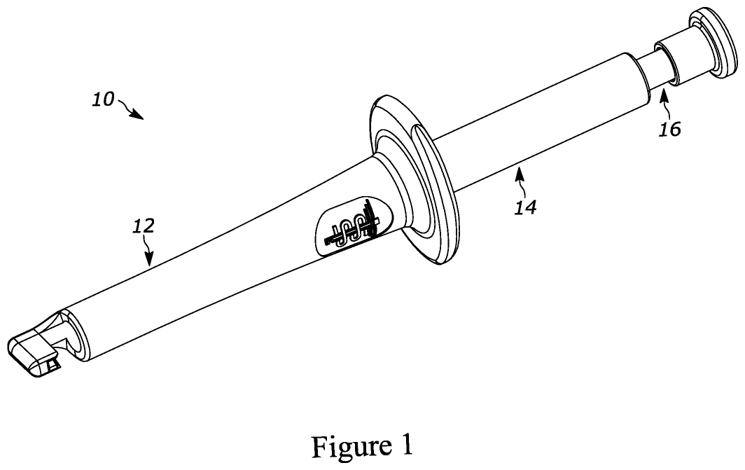

Referring now to the drawings and in particular to FIG. 1, suture cutting tool is shown generally at 10. While not limited thereto, the suture cutting tool is useful for the cutting of sutures that are internal to a body cavity. For example, during surgical procedures, it may become necessary to suture a lesion or the like within a body cavity, which suture will be encapsulated within the body of the patient. It is contemplated that the suture cutting tool may comprise a disposable tool or may comprise a tool that can be re-sterilized and reused in additional procedures. Furthermore, it is contemplated that the suture cutting tool is used to cut sutures of various sizes, including, but not limited to, #1 and #2 suture (USP), while other sizes are contemplated from, for example, 11-0 to 8 (usp), ranging in diameter from 0.01 to 1.09 mm. Of course, the suture cutting tool is not limited to use with such sutures.

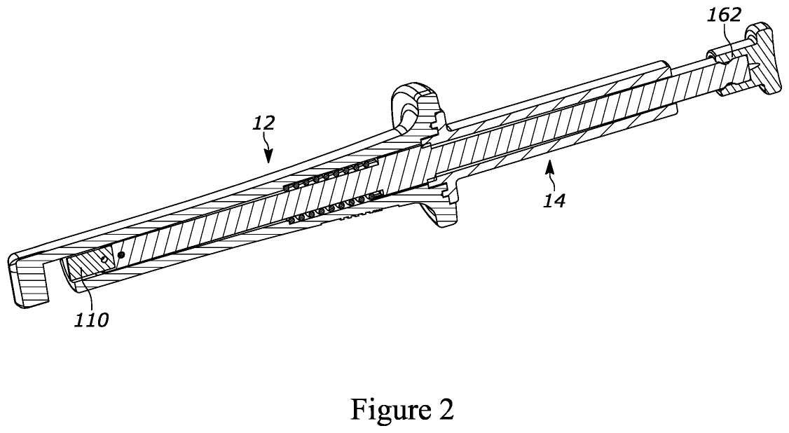

With reference to FIG. 1, the suture cutting tool 10 is shown as comprising housing 12, sleeve 14, and plunger assembly 16. As can be seen, and as will be described below, the plunger assembly is slidably movable within the housing and sleeve, which are coupled together in an end-to-end configuration. In the configuration of FIG. 1, the cutting tool is shown in its static state with no depression of the plunger button. A cross sectional view of the same is shown in FIG. 2.

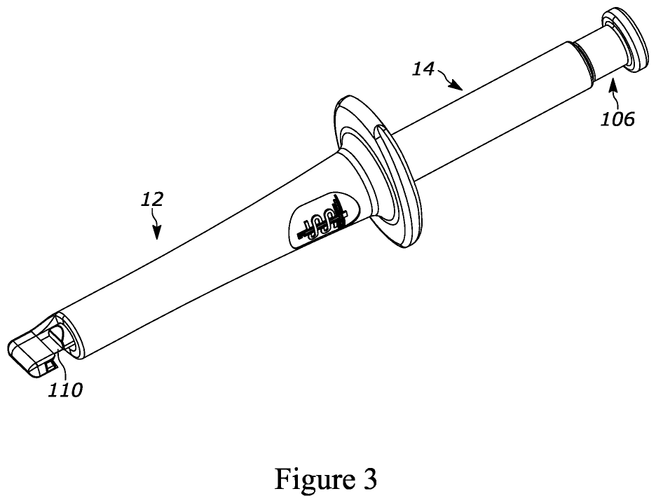

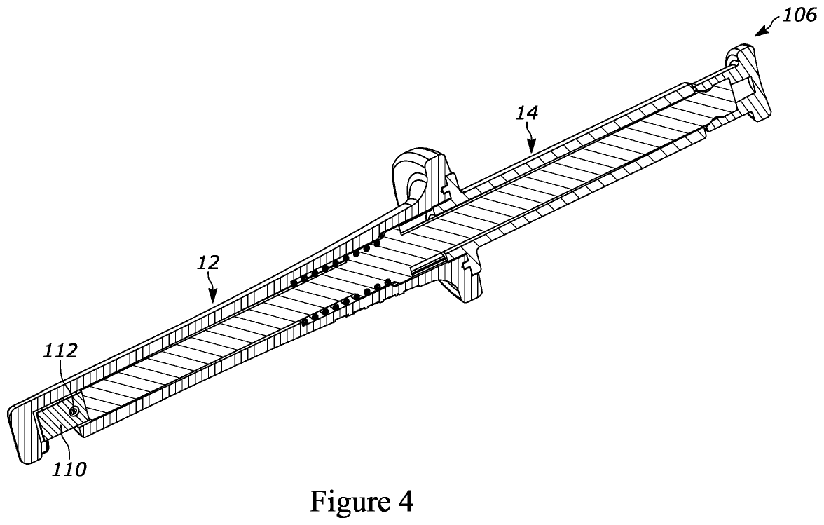

With reference to FIG. 3, suture cutting tool 10 is shown with the plunger button 106 depressed to slidably move the plunger rod through the housing and sleeve, thus exposing the cutting blade 110. A cross sectional view of the same is shown in FIG. 4. In the embodiment as shown, the housing comprises an integrally molded polymer member, and the sleeve comprises an integrally molded polymer member.

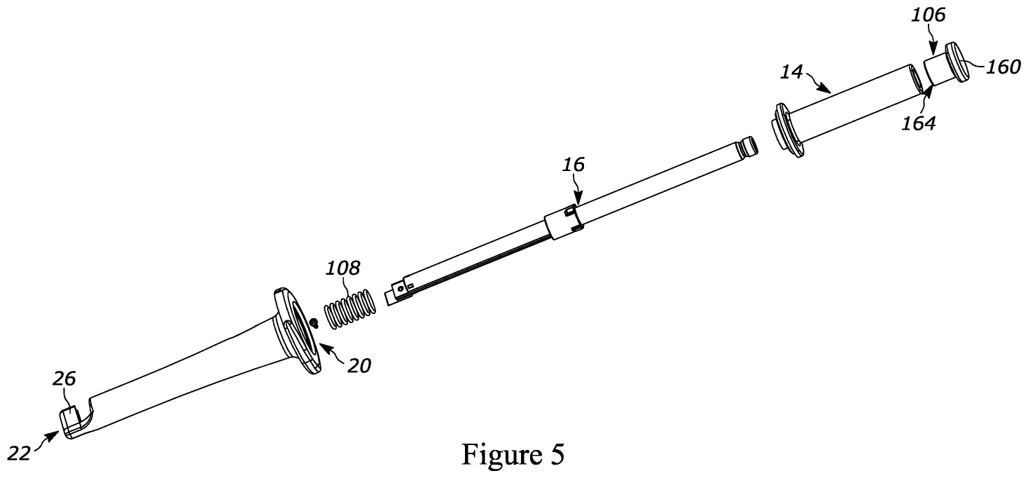

With reference to FIG. 5, housing 12 is shown as including proximal end 20 and distal end 22. Housing 12 may be formed from a single integrally molded polymer, while other materials (i.e., metals, composites) as well as other methods of formation (i.e., additive) are likewise contemplated. The anvil assembly 26 is positioned at the distal end of the housing 12. FIG. 5 further provides reference to sleeve 14, and plunger assembly 16.

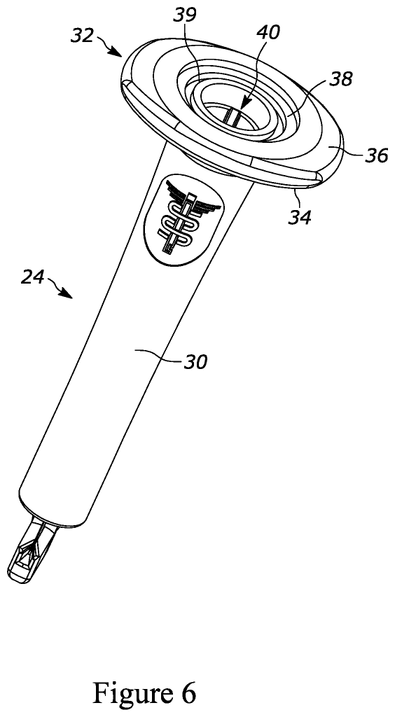

With reference to FIG. 6, a view of main body 24 is shown with an elongated configuration comprising an outer surface 30 and inner bore 40. The outer surface 30 includes flange 32 at the proximal end thereof. The flange is generally an outwardly directed configuration that extends about the outer surface 30, so as to provide a region by which to grasp the main body during operation. The flange includes lower surface 34 and upper side 36 opposite the lower surface 34. The lower surface 34 meets the remainder of the outer surface at a curved interface. The upper side 36 includes circumferential channel 38 that extends thereabout, and a further circumferential channel 39 defined within the inset surface formed by circumferential channel 38. These surfaces are configured, as will be described, for coupling of the sleeve 14 thereto.

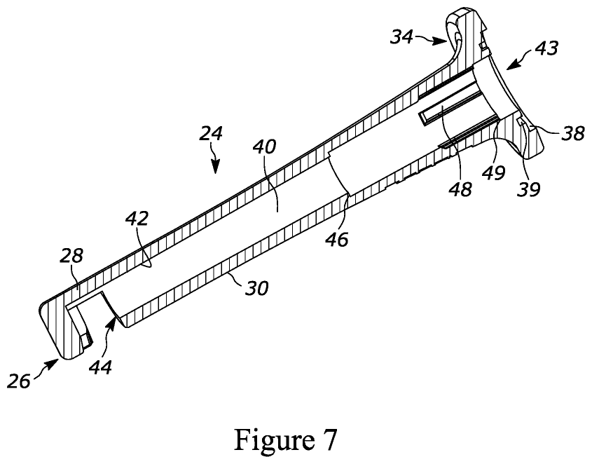

The inner bore 40 of the main body 24 is shown in greater detail in FIG. 7 as including inner bore 40 with wall surface 42, upper opening 43, and lower opening 44. Within the inner bore, wall surface 42 is shown as including lower shoulder 46, longitudinal slots 48 and upper shoulder 49. The inner bore 40 is sized to allow for sliding motion of the plunger assembly 16 therewithin. The upper opening 43 corresponds to the proximal end 20 of the housing 12. The inner bore 40 is generally cylindrical, while other configurations are contemplated, and defines a longitudinal axis therethrough.

The proximal end of inner bore 40, proximate the upper opening 43, has a larger cross-sectional area relative that reduces to define an upper shoulder 49 which, in the configuration shown is generally inwardly directed relative to wall surface 42. As will be described below, housing 12 is coupled to sleeve 14, upper shoulder 49 provides a contact surface to assist with limiting the inward insertion of the sleeve by interfacing with the distal end of sleeve 14. A lower shoulder 46 is defined between the upper and lower openings which is generally directed inwardly to wall surface 42 thereby reducing the cross-sectional area of the inner bore. Lower shoulder 46 provides a contact surface upon which return spring 108 may be compressed as the plunger rod 104 slides within the inner bore 40, as the return spring 108 is precluded from insertion therebeyond. In some configurations, the return spring may be omitted, and the user can manually move the plunger rod between the first and second positions (detents or the like may be formed at the first position to maintain the first position until the user desires to alter the first position).

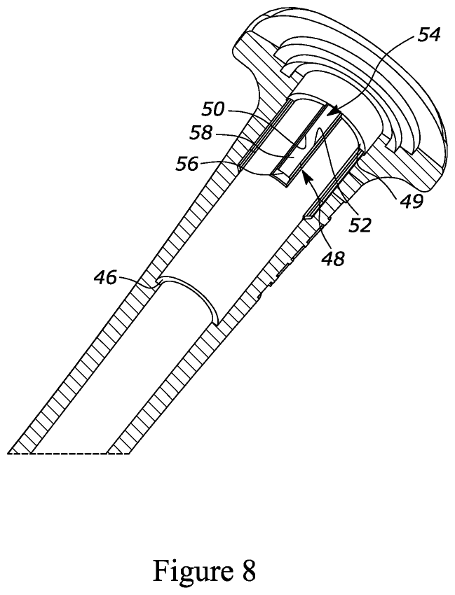

As shown in FIG. 8, a plurality of longitudinal slots 48 are disposed at predetermined annularly spaced intervals along the wall surface 42 of inner bore 40. Longitudinal slots 48 extend from upper shoulder 49 longitudinally along wall surface 42, defining entry region 54 and extending to a distal stop 56 spaced apart from the upper shoulder 49. Each of the plurality of longitudinal slots 48 is defined by a first sidewall 50, second sidewall 52, and bottom wall 58. The bottom wall 58 is tapered at the distal end to provide a sloped surface leading to distal stop 56. Additionally, the first and second sidewalls 50, 52 are inclined toward each other in the direction of the distal stop 56. In such a configuration, the outer surface tabs 139, while freely slidable, will become increasingly fixed (i.e., restricted from relative rotative movement) when reaching the distal stop 56. As will further be explained below, longitudinal slots 48 receive and allow for slidable motion of the outer surface tabs 139 of the plunger rod 104 therealong and assist in providing increasingly precise alignment of the plunger assembly within the inner bore 40 through the continued insertion of the plunger rod.

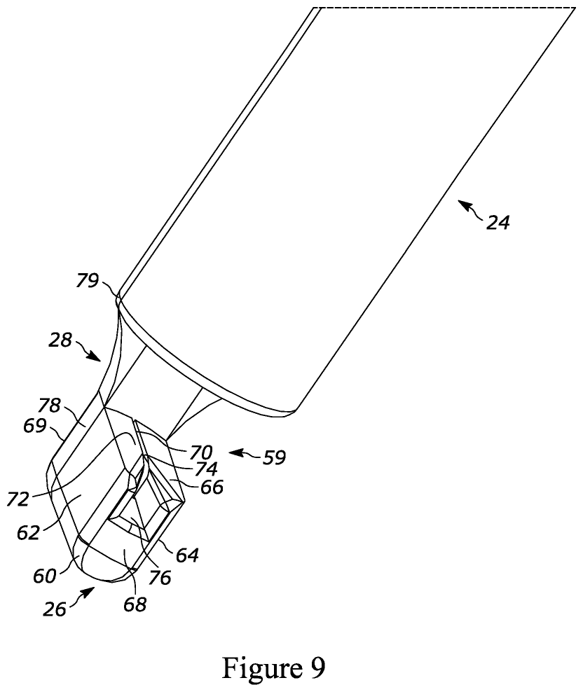

With reference to FIG. 9 anvil assembly 26 is shown spaced apart from the main body 24, and defining the distal end of housing 12. The connecting bridge 28 links the main body 24 to the anvil assembly 26. As will be explained below, the anvil provides a retention surface for the suture and further receives the cutting blade 110 upon passing through the suture when in use.

In greater detail, anvil assembly 26 is secured to main body 24 through connecting bridge 28. The connecting bridge 28 includes anvil end 78 which is coupled to the anvil assembly 26 and main body end 79 which is coupled to the distal end of the main body. As will be explained below, together, the anvil assembly 26, connecting bridge 28, and distal end 22 of main body 24 define a suture window 59, which allows for a material, such as a surgical suture, to be positioned across, through and/or within the suture window 59. The connecting bridge includes a facing surface which in the configuration show is generally perpendicular to the top wall of the anvil and parallel to the longitudinal axis. Preferably, the blade slot meets the facing surface with the spacing between the blade and the facing wall being minimized so as to preclude a suture from being wedged between the facing wall and the blade during use.

The anvil assembly 26 is shown in FIG. 9 as comprising distal surface 60 which generally defines the distal end 22 of the housing 12, and further comprises a first side surface 62 and second side surface 64, along with a front end 68 and back end 69. Back end 69 is coupled to the anvil end 78 of the connecting bridge 28. Anvil assembly 26 further comprises top surface 66, opposite to the upper opening 43 of inner bore 40, and generally facing the lower opening 44 of the inner bore. The top surface generally forms the distal surface of suture window 59.

A blade slot 70 is defined in the anvil assembly and extends away from the top surface 66 in a direction away from the lower end of the inner bore. The blade slot is defined by a first blade slot wall 72 and second blade slot wall 74, opposite the first blade slot wall. The blade slot walls are spaced apart from each other sufficiently to allow slidable insertion of a cutting blade 110 into blade slot 70. In the configuration shown, blade slot 70 is generally parallel to the longitudinal axis defined by the inner bore 40 of the main surface to allow for the slidable insertion of the blade, preferably, spaced apart from the first and second blade slot walls. In the configuration shown, the blade slot 70 is perpendicular to top surface 66 (and the first and second sidewalls meet the top wall generally with a minimal fillet, preferably so that the top wall meets the sidewalls so that they are perpendicular where meeting, or at an acute angle (that is portions of the sidewalls are within the footprint of the top wall, and the sidewalls move away from each other away from the top wall) of the anvil assembly and provides a relatively narrow passage that is sized to be larger than the blade, but sufficiently small so as to preclude the meaningful bending and/or meaningful introduction of the suture that is being cut into the blade slot 70. In the configuration as shown, blade 110 has a width of 0.004 inches with blade slot 70 having a passage width of 0.010 inches (i.e., preferably, the distance to each of the sidewalls is less than the thickness of the blade, while other configurations are contemplated). Thus, the blade is thicker than the spaces on either side of the blade (i.e., 0.003 is the space on either side and the blade is 0.004). It will further be understood that while the instant configuration renders a suture that is cut at the top wall (instead of being pushed into the blade slot), other dimensional relationships are contemplated, such as, for example, spaces between the blade and the slot walls that is less than the diameter of the suture being cut. For example, #1 suture has a diameter that is in the range of 0.400 and 0.499 mm, and #2 suture has a diameter that is in the range of 0.500 to 0.599 mm). Thus, the region on either side of the blade within the blade slot may be smaller than such diameters, for example. It will also be understood that the region on either side of the blade may be larger than the blade, while cutting the suture substantially proximate the top wall, instead of pushing the same into the blade slot. And by substantially on the top wall, it will be understood to one of skill in the art that the same would be sufficient not to tug on the suture, but to drive through the suture, while the regions over the slot may extend slightly into the slot, without pulling more suture than three times the width of the suture slot, for example, and preferably less than three times the width of the suture slot, and more preferably less than two times the width of the suture slot). Such movements of the suture will tend not to negatively impact the performance of the suture or damage the incision area.

Blade slot 70 is further defined by bottom wall 76 which spans between the first blade slot wall and the second blade slot wall. The bottom wall 76 forms the lower limit of movement of the blade 110 within blade slot 70. In the configuration shown, blade slot 70 may extend to and through the front end 68 of the anvil while the slot terminates proximate the connecting bridge 28.

In the configuration shown, anvil assembly 26 provides an anvil surface for placement and retention of the suture, so that the blade (which is described hereinbelow) can pass through the top surface 66 and further provides access for the blade into blade slot 70 so as to allow the blade to pass through the suture and to continue in the same direction upon cutting of the suture. In the embodiment shown, the width of blade slot 70 provides for a tight tolerance towards back end 69, so as to urge the cutting of the suture as opposed to pushing of the suture into the blade slot 70. Furthermore, by having the blade slot, the blade cutting surface is not dulled by interaction with the housing 12, but is allowed a location to be presented after cutting of the suture.

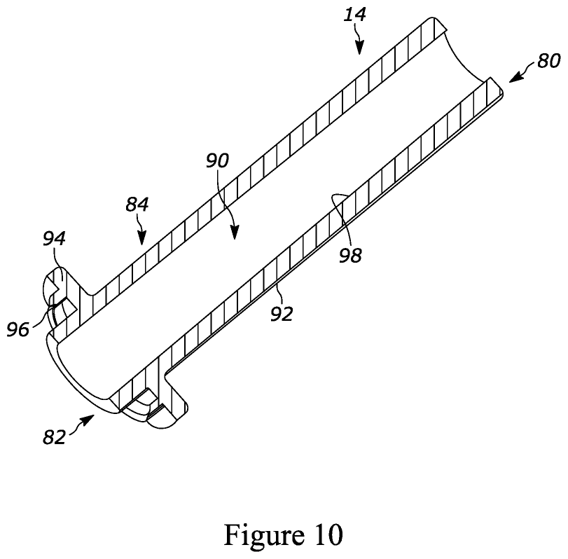

With reference to FIG. 10, sleeve 14 is shown as extending between a proximal end 80 and distal end 82, and defining elongated body 84. Sleeve 14 may be formed from a single integrally molded polymer, while other materials (i.e., metals, composites) as well as other methods of formation (i.e., additive) are likewise contemplated. As will be explained, sleeve 14 is coupled to the proximal end of the housing 12.

Elongated body 84 comprises outer surface 92, central bore 90, lower end tab 94, and circumferential ring tab 96. Central bore 90 defines an inner surface 98 which aligns with the longitudinal axis of the housing 12 and which is sized to allow for slidable motion of the plunger rod 104 therewithin. In the configuration shown, the inner surface 98 defines a generally uniform cross-sectional configuration, which, in the configuration shown, comprises a generally uniform circular cross-sectional configuration, thereby defining a cylindrical overall shape.

Proximate the distal end, the lower end tab 94 extends outwardly about the outer surface of the elongated body proximate the distal end 82. The lower end tab 94 includes an upper surface and a lower surface, with the lower surface being generally perpendicular to the longitudinal axis of the central bore. The circumferential ring tab 96 extends generally perpendicularly from the lower surface of the lower end tab 94 spaced apart from the outer surface of the elongated body 84. In the configuration shown, as the outer surface is generally cylindrical and the circumferential ring tab 96 is generally of a circular configuration, the outer surface and the circumferential ring tab are generally concentric about the longitudinal axis. Of course, other configurations are contemplated.

As will be explained below with respect to the assembly, lower end tab 94 is sized to fit within the circumferential channel 38 of the flange 32, and facilitates coupling between sleeve 14 and housing 12. At the same time, circumferential ring tab 96 fits within circumferential channel 39 of flange 32 to further facilitate coupling and alignment between sleeve 14 and housing 12. Finally, in such a coupled configuration, the distal end 82 of the elongated body 84 extends into inner bore 40 and engages upper shoulder 49 of the main body 24. Due to the relative sizing, the distal end 82 is precluded from passing beyond the upper shoulder 49.

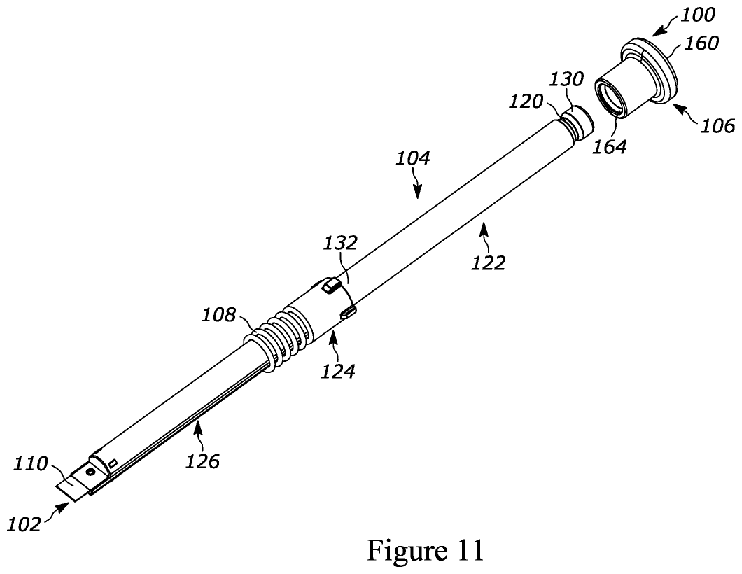

With reference to FIG. 11, plunger assembly 16 is shown as extending between proximal end 100 and distal end 102. Plunger assembly 16 includes plunger rod 104, plunger button 106, return spring 108, and blade 110. The plunger rod 104 and plunger button 106 may preferably be formed of molded polymer, while return spring 108 and blade 110 are preferably made of metal, though other appropriate construction materials are contemplated, such as polymer or composite based return springs, as well as metal or composite plunger rods and plunger buttons.

Plunger rod 104 is shown as comprising button coupling groove 120, upper shaft portion 122, central shaft portion 124, and lower shaft portion 126. The upper shaft portion includes proximal end 130 and distal end 132. The button coupling groove 120 is positioned at the proximal end 130 of the upper shaft portion, and is configured for attachment to the plunger button 106. As will be explained herein, the plunger rod 104 is configured to be slidably movable within the inner bore of the main body 24 of the housing 12 and the central bore of the elongated body of the sleeve 14.

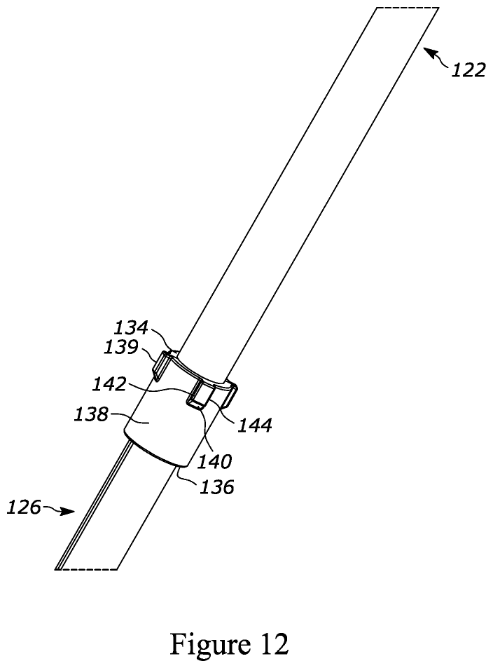

The Central shaft portion 124, as shown in FIG. 12, includes upper shoulder 134, lower shoulder 136, longitudinal outer surface 138, and outer surface tabs 139. Central shaft portion 124 is sized to have a greater diameter than upper shaft portion 122 and lower shaft portion 126, such that lower shoulder 136 is roughly the same circumference as lower shoulder 46 of inner bore 40, and the outer surface tabs 139 may slidably engage longitudinal slots 48 of inner bore 40. Outer surface tabs 139 as shown are annularly spaced around the central shaft portion to correspond with the spacing of longitudinal slots 48, and are defined by first tab side wall 142, second tab side wall 144, and lower end tab wall 140. In the present embodiment, the upper shaft portion 122 is integrally formed with the central shaft portion 124, but it is also contemplated that the two sections may be coupled together via alternate means, such as adhesives or threaded conjunction.

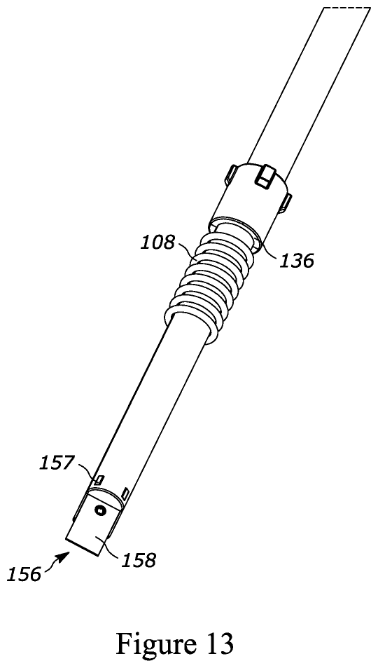

Lower shaft portion 126, as shown in FIG. 13, extends from the lower shoulder 136 to the distal end of the plunger rod 104, and includes the blade receiving region 156 and blade face surface 158. In the embodiment shown, blade face surface 158 defines a fastener receiving bore 159 to receive blade fastener 112 which secures the cutting blade to the plunger rod. In this embodiment, the blade fastener 112 is a threaded screw with blade opening 172 being threaded to receive the same. In some embodiments, the cutting blade may be secured through alternate means, such as an adhesive.

Lower shaft portion 126 is sized so that a return spring 108 may fully slide over the distal end and come in longitudinal contact with lower shoulder 136. The outer surface 154 of lower shaft portion 126 comprises protrusions 157 which are disposed near the distal end 102 but prior to blade receiving region 156. Protrusions 157 extend outwardly from outer surface 154 to interface with wall surface 42 as plunger rod 104 slides through housing 12. Contact between protrusions 157 and wall surface 42 as the blade reaches the top surface 66 of the anvil assembly ensures that the distal end of plunger rod 104 is in alignment so that the blade generally remains spaced apart from the first blade slot wall 72 and the second blade slot wall 74, and preferably centered therebetween. Due to the configuration of the inner bore of the main body of the housing, the protrusions 157 may remain spaced apart from or loosely connecting with the inner surface, and the interaction may increase as the blade reaches or approaches the top surface of the anvil assembly, as will be explained below. The engagement of the protrusions 157 with wall surface 42 of inner bore 40 further aligns the plunger rod 104 within the inner bore, which aligns the blade with the blade slot 70 of the anvil assembly.

Plunger button 106, as shown in FIG. 5, includes a top surface 160 which generally defines proximal end 100 of the plunger assembly and provides a surface for a user to apply pressure to push the plunger rod slidably through sleeve 14 and housing 12. Plunger button 106 is designed to allow for longitudinal coupling with proximal end 130 of the upper shaft portion. In the embodiment shown in FIG. 2, plunger button 106 comprises a coupling inlet 164 which is sized to allow for insertion of proximal end 130 therewithin, and further comprises an internal coupling ridge 162 within inlet 164. As currently shown, plunger rod 104 is constricted near the proximal end 130 to form a button coupling groove 120 which allows for plunger button 106 to snap into place overtop of proximal end 130, and allows plunger rod 104 and plunger button 106 to be coupled together through engagement of the button coupling groove 120 and internal coupling ridge 162. It is also contemplated that plunger button 106 may be coupled to the plunger rod through alternate means, such as adhesives, or may be integrally formed with the plunger rod 104. In the embodiment as shown, the distal end of plunger button 106 is sized to preclude insertion within sleeve 14 so as to prohibit further insertion of the plunger rod once the button is fully compressed.

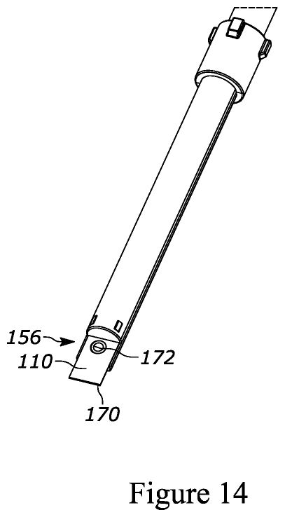

With reference to FIG. 14, blade 110 is shown as overlying the blade face surface 158 to allow the blade to be fastened to the blade receiving region by means of blade fastener 112. In the current embodiment, as discussed above, blade fastener 112 (FIG. 4) is a threaded screw which may be inserted through blade opening 172 and received into fastener receiving bore 159 of the blade receiving region. Blade 110 is sized to protrude beyond the distal end of the blade receiving region and have a width which facilitates slidable insertion of the blade into blade slot 70 of the anvil assembly, while being of a width which generally precludes scraping against or interfacing with the inner surface of the inner bore. Cutting edge 170 is located on the distal edge of blade 110 and is positioned to allow for insertion into blade slot 70 without contacting any surface of the anvil assembly 26, including bottom wall 76. In the configuration shown, the cutting edge is substantially perpendicular to the longitudinal axis of the plunger rod, while oblique, serrated or v-shaped configurations are contemplated.

In the embodiment as shown, suture cutting tool 10 may be assembled by first providing a blade and the plunger rod. The blade 110 is positioned to overlie the blade face surface 158 at the distal end of the plunger rod. Next, blade fastener 112 is directed through the blade opening 172 in the blade and into the fastener receiving bore 159 of the blade face surface so as to sandwich the blade between the face surface and fastener. Once secured, the return spring 108 is slid over the lower shaft portion 126 and upwardly moved so as to be proximate to lower shoulder 136 of the central shaft portion.

Once assembled, the plunger rod is ready for coupling with the housing 12. In particular, plunger rod 104 is slidably inserted into inner bore 40 of housing 12 by insertion through the upper opening 43 of the main body. As the insertion continues, the outer surface tabs 139 are slidably directed into a respective one of the longitudinal slots. Eventually, the return spring 108 contacts the lower shoulder 46 of the main body 24 so as to sandwich the return spring between the lower shoulder 46 and lower shoulder 136. At such time, further insertion compresses the return spring 108. Additionally, further insertion directs the outer surface tabs 139 toward the distal stop 56 of the longitudinal slots.

It will be understood that when the spring is not compressed, preferably, the blade remains within the inner bore so as to limit the ability of the blade to inadvertently cut or mar tissue or harm the user. Of course and as explained below, as the spring is compressed, the blade will extend beyond the lower opening 44 of the main body 24. This can be seen by comparing FIGS. 1 and 2 with FIGS. 3 and 4.

After plunger rod 104 has been inserted within housing 12, sleeve 14 is then positioned over top of the upper shaft portion of the plunger rod by slidably inserting proximal end 130 of upper shaft portion into central bore 90 of the sleeve. Housing 12 and sleeve 14 are fastened together by coupling flange 32 with distal end 82 of the sleeve, utilizing the associated circumferential ring tab 96, lower end tab 94, and the circumferential channels 38,39. In some embodiments, housing 12 and sleeve 14 are fastened by use of an adhesive, though other fastening structures are contemplated, including but not limited to heat welding, RF or ultrasonic welding, interference fits, snap fits and the like.

With the plunger rod 104 maintained within the coupled housing 12 and sleeve 14, the proximal end 100 protrudes from the sleeve central bore so as to allow plunger button 106 to be fastened to the plunger rod. In the embodiment as shown, the plunger button may snap over top of the proximal end of the plunger rod to facilitate engagement between the button coupling groove 120 and the internal coupling ridge 162. It is contemplated that plunger button 106 may also be fastened through alternate structures, such as use of adhesives or threaded fasteners, among others.

With the plunger assembly 16 fully assembled and appropriately positioned within the coupled housing 12 and sleeve 14, the configuration of suture cutting tool 10 is provided as a completed apparatus and as shown in FIG. 1. As previously discussed, FIG. 1 shows the suture cutting tool 10 prior to the application of force to depress plunger button 106 and to move the plunger and blade, as such, there compression of return spring 108 is in an extended configuration. In the preferred embodiment, blade 110 is completely retracted within inner bore 40 prior to application of a user actuated force upon to plunger button 106. This allows for the blade to remain protected within the housing body and helps to prevent accidental injury during handling of the tool.

To operate the suture cutting tool 10, a user generally positions a finger on either side of the housing 12, proximate to the lower surface of flange 32. The thumb of the user's same hand may be positioned to allow for depression of plunger button 106. Alternatively, the user may position the plunger button against the palm of their same hand and use their fingers to pull the cutting tool towards their palm, causing depression of the plunger button. In either instance, the tool may be operated by a single hand.

While holding the tool with a single hand, the user positions a suture, or other material to be cut, within the suture window 59. The suture should be positioned transverse across the top surface of the anvil such that blade 110 can be directed through the lower opening 44 of the housing main body, toward and into contact with the suture, through the suture and into the blade. To achieve the same, in the preferred operation, the suture should be positioned laterally across the top surface of the anvil and in direct contact with the top surface of the anvil assembly, such that the suture is supported on opposing sides of the blade slot during the cutting process. The user can pull on the suture to increase tension across the top wall.

Once the suture is properly positioned within the suture window and preferably across the top surface of the anvil assembly, the user depresses the plunger button to slidably move the plunger rod from the first position toward the second position through the housing and sleeve assembly and compress return spring 108. As the plunger rod moves through the housing, the blade receiving region 156 protrudes beyond lower opening 44, allowing the blade to cut through the suture and enter into blade slot 70 of the anvil, extending toward and into the second position.

To ensure proper alignment, so that the blade remains away from the top surface of the anvil and the side walls thereof, as the plunger button 106 is depressed, the plunger rod slides through housing 12 with the outer surface tabs 139 being directed slidingly through the longitudinal slots 48 defined on the wall surface of the inner bore 40. As described above, the longitudinal slots narrow towards the distal stop 56, wherein the outer surface tabs 139 more closely align with the sidewalls of the longitudinal slots, with less relative rotational motion. In greater detail, such a configuration helps to limit the radial motion of the plunger rod and provide precise alignment of the blade with the blade slot as the blade approaches the slot, while allowing for reduced force application to achieve slidable motion of the outer surface tabs through the longitudinal slots during initial movement prior to the blade approaching the slot.

While engagement between the surface tabs and longitudinal slots provides for proper radial alignment of the blade 110, the protrusions 157 slidably engage with the wall surface of the inner bore to provide optimal centering of the blade as it extends into the suture window through lower opening 44. This assists with translative movement of the blade relative to the housing.

In the preferred embodiment, the blade slot 70 is sized such that the blade cuts through the suture and enters the blade slot without leaving space on either side to allow the suture to bend-into or otherwise deform into the blade slot 70.

In some embodiments, the distance in which the plunger rod may be slidably moved through the housing is limited by the plunger button contacting the proximal end of sleeve 14. In other embodiments, the slidable longitudinal motion may be limited by the maximum compression of the return spring 108, or by contact between the outer surface tabs 139 and the distal end of the longitudinal slots 48. In the preferred embodiment, the maximum longitudinal motion of the plunger rod is achieved prior to the blade cutting edge contacting the bottom wall 76 of the blade slot. This prevents excess dulling of the blade during use, by contact of the blade with portions of the housing.

After the suture has been cut, the user releases pressure on the plunger button at which time the return spring biases the plunger rod in an opposing direction, namely, in a direction longitudinally back through the housing wherein the blade is retracted into the inner bore 40. The tool is then repositioned to cut the next suture or material. In other configurations, the user can manually return the plunger rod to toward first position.

It is contemplated that the tool may be designed and produced to be disposable after each surgical procedure. It is also contemplated that the tool may be designed and produced to be reusable with interchangeable parts and replaceable blades.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A suture cutting tool comprising:
a housing with a distal end, a proximal end, and an inner bore defined therethrough, the housing further comprising a main body having an anvil assembly, the anvil assembly positioned at the distal end of the housing, the main body and anvil assembly coupled by a connecting bridge to define a suture slot, the anvil assembly having top surface facing the inner bore and defining a blade slot between a first and second side of the top surface;
a plunger assembly having a plunger rod slidably movable within the inner bore of the housing with a first end extending beyond the housing and a second end extendable beyond the inner bore of the housing,
a blade coupled to a distal end of the plunger rod and including a cutting edge, the blade being movable along with the plunger assembly between at least a first position having the cutting edge within the suture slot to a second position having the cutting edge within the blade slot of the anvil;
whereupon movement between the first and second positions, the cutting edge is structurally configured to cut a suture traversing the blade slot within the suture slot.

2. The suture cutting tool of claim 1 wherein the cutting edge is precluded from reaching a lower end of a bottom wall of the blade slot.

3. The suture cutting tool of claim 1 wherein the blade slot further includes a first wall and a second wall opposite the first wall, the first and second walls being spaced apart from each other and depending from the top surface, wherein the blade is precluded from abutting either of the first and second walls.

4. The suture cutting tool of claim 1 wherein the blade defines a thickness and, upon insertion into the blade slot, a distance between the blade and the first wall and the blade and the second wall is less than or equal to the thickness of the blade.

5. The suture cutting tool of claim 4 wherein the blade has a thickness of 0.004″ and the blade slot has a width of 0.010″, with the blade substantially bisecting therebetween.

6. The suture cutting tool of claim 1 further including a return spring having a first end interfacing with the housing and a second end interfacing with the plunger rod, the return spring forcing the blade away from the second position.

7. The suture cutting tool of claim 1 wherein in the first position, the cutting edge is positioned within the inner bore of the housing.

8. The suture cutting tool of claim 1 further comprising:
at least one longitudinal slot defined into the inner bore of the housing terminating at the proximal end of the housing;
at least one outer surface tab on an outer surface of the plunger rod, the at least one outer surface tab slidably movable within the at least one longitudinal slot of the housing,
the at least one longitudinal slot capturing the at least one outer surface tab so as to constrain relative rotation of the plunger rod to the housing.

9. The suture cutting tool of claim 8 wherein the at least one outer surface tab and the at least one longitudinal slot are configured to increasingly constrain relative rotation of the plunger rod to the housing as the blade is directed from the first position to the second position.

10. The suture cutting tool of claim 8 wherein the at least one outer surface tab comprises four outer surface tabs and the at least one longitudinal slot comprises four longitudinal slots, space apart on the inner bore of the housing.

11. The suture cutting tool of claim 8 wherein the at least one outer surface tab has a lower end tab wall that interfaces with distal stop that precludes further movement in the direction of the second end.

12. The suture cutting tool of claim 8 wherein the outer surface of the plunger rod further includes a plurality of protrusions extending outwardly therefrom, the plurality of protrusions interfacing with the inner bore so as to improve alignment of the blade relative to the blade slot as the blade is directed to the second position.

13. The suture cutting tool of claim 12 wherein at least one of the plurality of protrusions contacts the inner bore as the blade is directed toward the second position prior to the cutting blade entering into the blade slot, while being spaced apart from the inner bore toward the first position.

14. The suture cutting tool of claim 1 wherein the plunger rod defines a longitudinal axis of travel within the inner bore, with the top surface of the anvil assembly being substantially perpendicular thereto.

15. The suture cutting tool of claim 1 wherein the blade slot includes a first sidewall and a second sidewall, with the first sidewall and second sidewall being parallel to each other and perpendicular to the top wall.

16. The suture cutting tool of claim 15 wherein the housing comprises two components coupled end to end, the first component being the main body and the second component being a sleeve, with the plunger rod extending through the main body and the sleeve.

17. The suture cutting tool of claim 1 wherein the connecting bridge further includes a facing wall, with the blade slot meeting the facing wall.

18. The suture cutting tool of claim 1 wherein the blade includes a cutting edge, with the cutting edge being substantially perpendicular to a longitudinal axis of the plunger rod.

19. A method of using a suture cutting tool comprising the steps of:
providing the suture cutting tool of claim 1;
placing a suture into the suture slot and across the blade slot;
directing the plunger rod from the first position to the second position; and
cutting the suture as the blade passes beyond the top wall.

20. A suture cutting tool comprising:
a housing having a proximal end and a distal end, with an internal bore extending therethrough, the distal end defining a suture slot that is transverse to the internal bore, with the housing terminating with an anvil assembly opposite the suture slot;
the anvil having a top wall extending thereacross with a blade slot depending from the top wall;
a plunger rod slidably positionable within the internal bore with a first end extending outside of the proximal end and a second end having a blade that is insertable into the suture slot and a cutting edge of which is insertable into the blade slot.
